(19) United States
(12) Patent Application Publication
(10) Pub. No.: US 2024/0423956 A1
(43) Pub. Date: Dec. 26, 2024

(54) METHOD FOR ADMINISTERING A FORMULATION TO AN ANIMAL

(71) Applicant: Tevra Brands, LLC, Omaha, NE (US)

(72) Inventor: Caryn Stichler, Omaha, NE (US)

(21) Appl. No.: 18/825,860

(22) Filed: Sep. 5, 2024

Related U.S. Application Data
(63) Continuation of application No. 18/103,193, filed on Jan. 30, 2023.
Application No. 18/103,193 is a continuation-in-part of application No. 18/090,560, filed on Dec. 29, 2022.
Application No. 18/090,560 is a continuation-in-part of application No. 17/105,748, filed on Nov. 27, 2020, now abandoned.
(60) Provisional application No. 62/943,556, filed on Dec. 4, 2019.

(51) Int. Cl. A61K 31/4045; A61K 9/00; A61K 35/12; A61K 47/24; A61K 47/26; A61K 47/54

(52) U.S. Cl. CPC A61K 31/4045; A61K 9/006; A61K 35/12; A61K 47/24; A61K 47/26; A61K 47/543

(57) ABSTRACT

A method for administering a formulation to an animal involves using a dispenser that includes a top end with one or more openings and a bottom end equipped with a tightening mechanism. The method comprises the steps of contacting the formulation application area with the top end of the dispenser and administering the formulation by adjusting the tightening mechanism. This adjustment pushes the formulation through the openings in the top end, directing it onto the formulation application area. The described method ensures controlled and efficient delivery of the formulation to the intended site on the animal.

### CROSS-REFERENCE TO RELATED APPLICATIONS

This Continuation application claims the benefit of Continuation-in-Part U.S. patent application Ser. No. 18/103,193 filed Jan. 30, 2023, which claims the benefit of Continuation-in-Part U.S. patent application Ser. No. 18/090,560 filed Dec. 29, 2022, which claims the benefit of U.S. patent application Ser. No. 17/105,748 filed Nov. 27, 2020, which claims the benefit of U.S. Provisional Patent Application No. 62/943,556 filed Dec. 4, 2019, each of which is hereby incorporated by reference.

### STATEMENT REGARDING FEDERALLY SPONSORED RESEARCH

Not Applicable.

### APPENDIX

Not Applicable.

### BACKGROUND OF THE INVENTION

### Field of the Invention

The present invention relates to a melatonin-containing transmucosal gel for oral ingestion for dogs, and more particularly to a melatonin-containing transmucosal gel for oral ingestion dogs that provides calming actives in an easily administered dispenser.

### Related Art

Canine anxiety (anxiety) is a response to fear and agitation, or apprehension when the dog anticipates a threat or fearful situation. Some individual dogs experience disproportionate levels of anxiety. Anxiety can develop into an anxiety disorder and can lead to behavioral and other issues. Anxiety can take the form of one of various anxiety disorders such as generalized anxiety disorders, excessive stimulus anxiety, separation anxiety, confinement, noise phobias, among others.

Causative factors may include genetic components, prenatal and neonatal stressors, maternal separation, lack of socialization, unfamiliarity, or a previous unpleasant outcome during encounters with the stimulus (or similar stimuli). The most common causes are fear, separation and aging. Fear-related anxiety can be caused by loud noises, strange people or animals, visual stimuli, new or strange environments, and specific situations among others. Age-related anxiety affects older dogs and can be associated with cognitive dysfunction syndrome (CDS). Separation anxiety is a specific anxiety arising from an inability of the pet to find comfort when separated from family members. Anxiety may lead to destructive behavior (particularly at exits or toward owner possessions), distress vocalization, house-soiling, salivation, pacing, restlessness, inability to settle, anorexia, and repetitive or compulsive behaviors. In some instances, anxiety may play a role in aggressive behavior.

Common symptoms of dog anxiety include aggression, urinating or defecating in the house, drooling, panting, destructive behavior, depression, excessive barking, pacing, restlessness and repetitive or compulsive behaviors. Different dogs display different symptoms and combinations of symptoms when suffering from anxiety.

Canine stress is the response of dog to a demand placed upon it to change or adapt, typically exhibited as feelings of strain or pressure. Dogs experiencing stress may result in feelings of fear, agitation, hyperactivity, nervousness, oversensitivity or irritability. Negative stress, excessive stress and chronic stress can have a detrimental effect on behavior, health and overall well-being. Stress has the potential to bring on illness, suppress the immune system, cause undesirable behaviors, and increase arousal, which increases the probability of aggressive behavior.

Causes of stress in dogs include grief, exposure to conflict, excessive or insufficient stimulation, overcrowded conditions, environmental changes (schedule, people, animals, increased noise); punitive training, insufficient social time, frightening events, neglect, frustration, and uncertainty among others.

There is a need for methods and compositions for reducing stress and anxiety in canines. Melatonin has been found to be useful in treating stress and anxiety. However, melatonin treatments encounter several problems. Melatonin is eliminated from the blood quickly once administered. When administered orally, melatonin has limited solubility in water and shows low and variable bioavailability. As with most oral administrations, it can take more than 30 minutes after ingestion for the blood plasma concentration of melatonin to reach its peak.

U.S. Pat. No. 5,362,745 discloses an oral pharmaceutical composition consisting of melatonin as the active principle in form of a micro-emulsion using L-α-phosphatidylcholine as an emulsifier and a solvent mixture consisting of ethanol, propylene glycol and water and wherein the weight ratio of L-α-phosphatidylcholine to melatonin is about 1:1 and wherein the composition optionally contains a thickener agent. The patent fails to disclose trans-mucosal administration of melatonin.

Sublingual, buccal, orally dissolving tablets and films containing melatonin are known. For example, transmucosal formulations are described in WO 1996/030013 and U.S. Pat. No. 5,688,520. However, in these formulations, melatonin is compounded in its undissolved, or solid, state. For any drug to be absorbed into the bloodstream, it must be dissolved, i.e., in solution. Due to melatonin's poor water solubility much of the dosage from a currently available preparation is swallowed undissolved in the saliva, leading to poor and erratic absorption in the GI tract. Accordingly, melatonin, having low to poor water solubility, is expected to be poorly suited for buccal or sublingual administration.

Another route of administration for melatonin using nasal and oral sprays has been disclosed in U.S. Pat. No. 6,007,834. However, sprays are less desirable because of improper manipulation of the actuator, swallowing of the dosage before dissolution of the drug, and the restrictions on usage when the patient has sinus congestion or a head cold.

U.S. Pat. No. 8,992,948 discloses the buccal and sublingual administration of melatonin prepared by dissolving the melatonin in a pharmaceutically acceptable solvent where the solvent is selected from polyethylene glycol, ethanol, ethyl acetate, isopropyl alcohol, triacetin, triethyl citrate, tributyl citrate, substituted polyethylene glycols, propylene glycol, bisabolol, glycerin, mineral oil, oleic acid, oleyl alcohol, ethyl oleate, fatty acid esters, squalane, animal oils, vegetable oils, hydrogenated vegetable oils, isopropyl myristate, isopropyl palmitate, glycofurol, terpenes, essential oils, alcohols, water, polyols, silicone fluids, and/or glycerides and mixtures thereof. The melatonin solution is absorbed onto a solid carrier, and thereafter formed into a solid dosage form such as a tablet. The carrier used in the subject composition is preferably a pharmaceutically acceptable carrier provided as a bead, a granule, a particle, or the like. When administered, the tablet is held under the tongue.

Other calming formulations do not use melatonin. For example, U.S. Pat. No. 6,255,341 discloses an edible solid composition containing Vitamin C, Vitamin E, selenium, β-carotine, triglycerides and protein to reduce stress. U.S. Pat. No. 11,337,958 discloses the use of Vitamin C and soy protein to reduce stress in canines.

Pheromone compositions mimic certain naturally occurring pheromones that have a calming effect on mammals. As such, the compositions are useful for controlling behavior in mammals and are especially useful for controlling stress- or anxiety-induced behavior in pets and domestic animals. Pheromones are thought to be detected by olfactory membranes or by the vomeronasal organ (VNO or Jacobson's organ), which is positioned between the nose and mouth and functions as the first stage of the accessory olfactory system. However, unlike regular olfactory membranes, the VMO is connected directly to the mid-brain and thus enjoys the shortest organ-to-brain distance in mammals. This feature allows pheromones present in extremely small quantities to very selectively trigger certain biochemical processes in the animal.

Pheromones are administered as edible compositions when used as bait for attracting and controlling pests such as rodents and insects. However, olfactory compositions are typically used to administer calming compositions. Quinolone, as disclosed in U.S. Patent Application 2019/0,269,615 is a pheromone with calming properties. U.S. Pat. No. 8,481,020 discloses additional pheromones.

None of the above-cited references disclose the claimed gel composition and the trans-mucosal method of administering melatonin, and optionally, a pheromone, to a canine for calming.

### SUMMARY OF THE INVENTION

The calming gel of the present invention suitable for canine use contains from about 4 wt. % to about 33 wt. % lipid; from about 8 wt. % to about 29 wt. % phospholipid surfactant; from about 34 wt. % to about 60 wt. % humectant; from about 1.0 to 0.001 wt. % melatonin; and from about 1.0 to about 10.0 wt. % calming ingredients. In a preferred embodiment, the calming gel contains a pheromone at less than about 0.001 wt. %. The calming gel is homogeneous and hydrophobic.

In a preferred embodiment, the calming gel contains from about 4 wt. % to about 5 wt. % petrolatum; from about 8 wt. % to about 12 wt. % lecithin; from about 28 wt. % to about 35 wt. % sorbitol; from about 10 wt. % to about 25 wt. % glycerin; from about 1.0 to 0.001 wt. % melatonin; and from about 1.0 to about 10.0 wt. % calming agents. The calming gel does not contain breath freshening agents such as eucalyptol, methyl salicylate, thymol, menthol, oil of wintergreen, oil of peppermint, oil of spearmint, clove bud oil, anethole, methyl salicylate, cassia, 1-menthyl acetate, sage, eugenol, parsley oil, oxanone, alpha-irisone, marjoram, lemon, orange, propenyl guaethol, cinnamon, vanillin, linalool, cinnamaldehyde glycerol acetal known as CGA, and mixtures thereof. In a preferred embodiment, the calming gel is alcohol-free. In another preferred embodiment, the calming gel contains from about 0.001 wt. % to about 0.000001 wt. % pheromone.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DESCRIPTION OF THE FIGURE

FIG. 1 is a top view of a dispenser of the calming gel of the present invention.

### DETAILED DESCRIPTION OF THE PREFERRED EMBODIMENTS

The following description of the preferred embodiment(s) is merely exemplary in nature and is in no way intended to limit the invention, its application, or uses.

The lipids of the present invention may be of animal, vegetable or mineral origin, which are substantially water-insoluble, inert, non-toxic hydrocarbon fats and oils and derivatives thereof, and may comprise any of the commonly commercially available fats or oils approved by the Food & Drug Administration, having melting points in the range of about 90 to 160° F. The lipid may comprise a vegetable oil base commonly known as hard butter. Hard butters are hydrogenated, press fractionated or other processed oils that are processed or recombined to have a solid fat index (percent solid fat vs. temperature) similar to that of cocoa butter. However, other lipids may be used that are relatively hard or solid at room temperature, but melt rapidly in the mouth at a temperature of about 98° F. (mouth temperature).

Examples of suitable lipids include tallow, hydrogenated tallow, hydrogenated vegetable oil, almond oil, coconut oil, corn oil, cottonseed oil, light liquid petrolatum, heavy liquid petrolatum, olein, olive oil, palm oil, peanut oil, persic oil, sesame oil, vitamin E, soybean oil or safflower oil. Additionally, stearines can be used as a lipid in the present invention. The addition of stearines to the product provides the favorable property of mold-release. Further, the addition of stearines raises the melting point of the composition as high as about 100° F., which is particularly beneficial when the product is shipped or stored in unrefrigerated compartments.

Preferred examples include petrolatum, a mineral oil (Vaseline oil), which may be any petroleum based product; modified or unmodified vegetable oils such as peanut oil, wheat germ oil, linseed oil, jojoba oil, apricot kernel oil, walnut oil, palm oil, pistachio oil, sesame oil, colza oil, cade oil, corn germ oil, peach kernel oil, poppy seed oil, soya oil, safflower oil, coconut oil, hazelnut oil, grapeseed oil, avocado oil, soy oil, sweet almond oil, calophyllum oil, olive oil, sunflower oil, or animal oils such as whale oil, seal oil, menhaden oil, halibut liver oil, cod liver oil, cod, tuna, turtle tallow, horse's hoof, sheep's foot, mink, otter, marmot oil and the like; synthetic oils such as silicon oil such as dimethylpolysiloxane; alkyl and alkenyl esters of fatty acids, such as isopropyl esters of myristic, palmitic and stearic acids and fatty esters which are solid at room temperature; waxes such as lanolin wax, candelilla wax, spermaceti, cocoa butter, karite butter, silicon waxes, hydrogenated oils which are solid at room temperature, sucro-glycerides, oleates, myristates, linoleates, stearates, paraffin, beeswax, carnauba wax, ozokerite, candelilla wax, microcrystalline wax; fatty alcohols such as lauryl, cetyl, myristyl, stearyl, palmityl and oleyl alcohols; polyoxyethylated fatty alcohols; and wax esters, lanolin and its derivatives, perhydrosqualene and saturated esters, ethyl palmitate, isopropyl palmitate, alkyl myristates such as isopropyl myristate, butyl myristate and decyl myristate, hexyl stearate, triglyceride esters, triglycerides of octanoic and decanoic acid, cetyl ricinoleate, stearyl octanoate (Purcellin oil), fatty acids, polyhydric alcohols, polyether derivatives, fatty acid monoglycerides, and hydrogenated palm oil (Paramount XX), and mixtures of waxes and oils. The preferred lipids are a combination of hydrogenated palm oil and vitamin E. The preferred calming gel contains from about 4 wt. % to about 33 wt. % petrolatum. The more preferred calming gel contains from about 4 wt. % to about 5 wt. % petrolatum.

The surfactant suitable for the present canine and feline product selected from phospholipids. Other well-known surfactants are not suitable for consumption by dogs and cats. Suitable phospholipids include any of a group of phospholipids occurring in animal and plant tissues and egg yolk, composed of units of choline, phosphoric acid, fatty acids, and glycerol. This includes a commercial form of phospholipids obtained chiefly from soybeans, corn, and egg yolk, specifically including lecithin. The preferred calming gel contains from about 9 wt. % to about 29 wt. % lecithin. The more preferred calming gel contains from about 9 wt. % to 12 wt. % lecithin.

Suitable humectants include, but are not limited to glycerin, lactic acid, polyols, propylene glycol, corn syrup, high fructose corn syrup (HFCS), including Cornsweet 55 and Cornsweet 42, and sorbitol. The preferred form of humectant is a combination of non-crystallizing liquid sorbitol (70 wt. % sorbitol in water) and glycerine. The preferred calming gel has about 34 to 60 wt. % humectant. The more preferred calming gel has from about 14 wt. % to about 35 wt. % sorbitol and from about 20 wt. % to about 25 wt. % glycerin. In a preferred embodiment, the calming gel has a ratio of sorbitol to glycerine of about 1:2 to 2:1.

Calming ingredients for ingestion include melatonin, thiamine, chamomile, valerian, niacin and L-tryptophan. Other ingredients include chamomile, lavender, Saint John's Wort, Kawakawa, valerian, passionflower, γ-aminobutyric acid, serotonin, cedrol, vitamin E, selenium, vitamin C, β-Carotene, other vitamins, minerals, supplements, and additional ingredients.

Pheromones suitable for the present invention include individual pheromones and blends of pheromones, which contain oleic acid (62.34 wt. %), palmitic acid (19 wt. %), pimelic acid (9 wt. %), azelaic acid (9 wt. %), butylated hydroxytolune (0.33 wt. %), and salicylic acid (0.33 wt. %). Additionally, quinolone, as disclosed in U.S. Patent Application 2019/0,269,615 is a pheromone with calming properties. Tiglic aldehyde, sold by Sigma Aldrich, is a pheromone comprises tiglinaldehyde and is used as a rabbit pheromone.

Additional ingredients are included in the canine calming gel, such as flavorings and palatants.

The samples of the invention were made by the following process:

Examples 1-11 were prepared as follows:
1. Weigh all petrolatum and lecithin directly into Kitchen Aid precise heat mixing bowl.
2. Turn heat setting to 165° F.
3. Attach the blade and begin slow mixing.
4. When product temperature reaches at least 160° F., turn heat OFF.
5. In a suitable bowl or beaker, add glycerin and flavoring. Warm to 130° F.
6. Add melatonin, mix well.
7. Add thiamine-mix well.
8. Add chamomile-mix well.
9. Add valerian-mix well.
10. Add milled L-Tryptophan-mix well.
11. In a laboratory beaker, weigh the water.
12. Weigh and add the niacin, while stirring, until dissolved (minimum 15 minutes).
13. Once the niacin is dissolved, add sorbitol to form a sorbitol solution and mix well.
14. Add the sorbitol solution to the glycerin solution and mix for 5 minutes.
15. Add the pheromone to the sorbitol/glycerin solution and mix for 1 minute.
16. Gradually add the sorbitol/glycerin solution to the petrolatum/lecithin mix in the Kitchen Aid precise heat bowl, while stirring continuously.
17. Maintain a product temperature of 130° F. during the process.
18. With the heat turned OFF, allow to cool while continuously stirring.
19. When the product temperature cooled to 110° F., pour into storage containers.

The calming gel is hydrophobic, homogenous and stable.

The pheromone blend of Examples 4-9 contains:
oleic acid (62.34 wt. %),
palmitic acid (19 wt. %),
pimelic acid (9 wt. %)
zelaic acid (9 wt. %)
butylated hydroxyl toluene (BHT) (0.33 wt. %)
BHA (0.33 wt. %)

76-97 Canine Calming Gel with Quinoline Pheromone

| RM # | Ingredient | Weight | wt. % |
|---|---|---|---|
| 996 | Yelkin ® Gold - Lecithin | 18.80 | 9.40% |
| 445 | citation 70 - Mineral Oil | 9.20 | 4.60% |
| 1820 | Beef Ty Fl NAT - Flavoring | 2.00 | 1.00% |
| 1796 | Melatonin | 0.092 | 0.046% |
| 1544 | Quinoline - Pheromone | 0.0002 | 0.0001% |
| 1801 | Niacin, USP | 0.40 | 0.20% |
| 1821 | Thiamine HCl Vitamin B1 | 0.70 | 0.35% |
| 1807 | Chamomile Extract | 9.40 | 4.70% |
| 1805 | Valerian Root Extract | 6.00 | 3.00% |
| 1811 | L-Tryptophan | 1.80 | 0.90% |
| 997 | Sorbitol Solution 70% USP | 80.00 | 28.00% Sorbitol, |
| | | | 12% water |
| 995 | Glycerin, USP 99.7% Excipient/ | 24.00 | 12.00% |
| | Food use | | |
| 529 | purified water (Distilled) | 47.6078 | 23.8039% |

76-98 Canine Calming Gel - No Pheromone

| RM # | Ingredient | Weight | wt. % |
|---|---|---|---|
| 996 | Yelkin ® Gold - Lecithin | 19.00 | 9.50% |
| 445 | citation 70 - mineral oil | 8.00 | 4.00% |
| 1820 | Beef Ty Fl NAT - flavoring | 3.00 | 1.50% |
| 1796 | Melatonin | 0.086 | 0.043% |
| 1801 | Niacin, USP | 0.344 | 0.172% |
| 1821 | Thiamine HCl Vitamin B1 | 0.69 | 0.345% |
| 1807 | Chamomile Extract | 2.00 | 1.00% |
| 1805 | Valerian Root Extract | 1.00 | 0.50% |
| 1811 | L-Tryptophan | 1.72 | 0.86% |
| 997 | Sorbitol Solution 70% USP | 80.00 | 40.00% |
| 995 | Glycerin, USP 99.7% Excipient/Food use | 44.16 | 22.08% |
| 529 | purified water (Distilled) | 40.00 | 20.00% |

76-99 Canine Calming Gel with Quinoline Pheromone

| RM # | Ingredient | Weight | wt. % |
|---|---|---|---|
| 996 | Yelkin ® Gold Lecithin | 19.00 | 9.50% |
| 445 | citation 70 - Mineral Oil | 8.00 | 4.00% |
| 1820 | Beef Ty Fl NAT - Flavoring | 3.00 | 1.50% |
| 1796 | Melatonin | 0.086 | 0.043% |
| 1544 | Quinoline - Pheromone | 0.00012 | 0.00006% |
| 1801 | Niacin, USP | 0.344 | 0.172% |
| 1821 | Thiamine HCl Vitamin B1 | 0.69 | 0.345% |
| 1807 | Chamomile Extract | 2.00 | 1.00% |
| 1805 | Valerian Root Extract | 1.00 | 0.50% |
| 1811 | L-Tryptophan | 1.72 | 0.86% |
| 997 | Sorbitol Solution 70% USP | 80.00 | 28.00% sorbitol, |
| | | | 12% water |
| 995 | Glycerin, USP 99.7% Excipient/ | 44.16 | 22.08% |
| | Food use | | |
| 529 | purified water (Distilled) | 39.99976 | 19.99988% |

78-10 PC76-99 Canine Calming Gel with Pheromone Blend

| RM # | Ingredient | Weight | wt. % |
|---|---|---|---|
| 996 | Yelkin ® Gold - Lecithin | 47.50 | 9.50% |
| 445 | citation 70 - Mineral Oil | 20.00 | 4.00% |
| 1820 | Beef Ty Fl NAT - Flavoring | 7.50 | 1.50% |
| 1796 | Melatonin | 0.215 | 0.043% |
| 1822 | Pheromone Blend | 0.00030 | 0.00006% |
| 1801 | Niacin, USP | 0.860 | 0.172% |
| 1821 | Thiamine HCl Vitamin B1 | 1.73 | 0.345% |
| 1807 | Chamomile Extract | 5.00 | 1.00% |
| 1805 | Valerian Root Extract | 2.50 | 0.50% |
| 1811 | L-Tryptophan | 4.30 | 0.86% |
| 997 | Sorbitol Solution 70% USP | 200.00 | 28.00% sorbitol, |
| | | | 12% water |
| 995 | Glycerin, USP 99.7% Excipient/ | 110.40 | 22.08% |
| | Food use | | |
| 529 | purified water (Distilled) | 99.99940 | 19.99988% |

78-14 PC76-99 Canine Calming Gel with Pheromone Blend

| RM # | Ingredient | Weight | wt. % |
|---|---|---|---|
| 996 | Yelkin ® Gold - Lecithin | 47.50 | 9.50% |
| 445 | citation 70 - Mineral oil | 20.00 | 4.00% |
| 1820 | Beef Ty Fl NAT - Flavoring | 7.50 | 1.50% |
| 1796 | Melatonin | 0.215 | 0.043% |
| 1822 | Pheromone Blend | 0.00030 | 0.00006% |
| 1801 | Niacin, USP | 0.860 | 0.172% |
| 1821 | Thiamine HCl Vitamin B1 | 1.73 | 0.345% |
| 1807 | Chamomile Extract | 5.00 | 1.00% |
| 1805 | Valerian Root Extract | 2.50 | 0.50% |
| 1811 | L-Tryptophan | 4.30 | 0.86% |
| 997 | Sorbitol Solution 70% USP | 200.00 | 28.00% sorbitol, |
| | | | 12% water |
| 995 | Glycerin, USP 99.7% Excipient/ | 110.40 | 22.08% |
| | Food use | | |
| 529 | purified water (Distilled) | 99.99940 | 19.99988% |

78-29 PC78-10 Canine Calming Gel with Pheromone Blend

| RM # | Ingredient | Weight | % |
|---|---|---|---|
| 996 | Yelkin ® Gold - Lecithin | 47.50 | 9.50% |
| 445 | citation 70 - Mineral oil | 20.00 | 4.00% |
| 1820 | Beef Ty Fl NAT - Flavoring | 7.50 | 1.50% |
| 1796 | Melatonin | 0.215 | 0.043% |
| 1822 | Pheromone Blend | 0.00030 | 0.00006% |
| 1801 | Niacin, USP | 0.860 | 0.172% |
| 1821 | Thiamine HCl Vitamin B1 | 1.73 | 0.345% |
| 1807 | Chamomile Extract | 5.00 | 1.00% |
| 1805 | Valerian Root Extract | 2.50 | 0.50% |
| 1811 | L-Tryptophan | 4.30 | 0.86% |
| 997 | Sorbitol Solution 70% USP | 200.00 | 28.00% sorbitol, |
| | | | 12% water |
| 995 | Glycerin, USP 99.7% Excipient/ | 110.40 | 22.08% |
| | Food use | | |
| 529 | purified water (Distilled) | 99.99940 | 19.99988% |

78-30 PC78-10 Canine Calming Gel with Pheromone Blend

| RM # | Ingredient | Weight | wt. % |
|---|---|---|---|
| 996 | Yelkin ® Gold - Lecithin | 14.25 | 9.50% |
| 445 | citation 70 - Mineral oil | 6.00 | 4.00% |
| 1822 | Pheromone Blend | 0.0001 | 0.00006% |
| 1820 | Beef Ty Fl NAT | 2.25 | 1.50% |
| 1796 | Melatonin | 0.0645 | 0.043% |
| 1801 | Niacin, USP | 0.258 | 0.172% |
| 1821 | Thiamine HCl Vitamin B1 | 0.5175 | 0.345% |
| 1807 | Chamomile Extract | 1.50 | 1.00% |
| 1805 | Valerian Root Extract | 0.75 | 0.50% |
| 1811 | L-Tryptophan | 1.29 | 0.86% |
| 997 | Sorbitol Solution 70% USP | 60.00 | 28.00% sorbitol, |
| | | | 12% water |
| 995 | Glycerin, USP 99.7% Excipient/ | 33.12 | 22.08% |
| | Food use | | |
| 529 | purified water (Distilled) | 29.9997 | 19.99982% |

78-76 PC78-10 Canine Calming Gel with Pheromone Blend

| RM # | Ingredient | Weight | wt. % |
|---|---|---|---|
| 996 | Yelkin ® Gold - Lecithin | 14.25 | 9.50% |
| 445 | citation 70 - Mineral oil | 6.00 | 4.00% |
| 1822 | Pheromone Blend | 0.0001 | 0.00006% |
| 1820 | Beef Ty Fl NAT - Flavoring | 2.25 | 1.50% |
| 1796 | Melatonin | 0.0645 | 0.043% |
| 1801 | Niacin, USP | 0.258 | 0.172% |
| 1821 | Thiamine HCl Vitamin B1 | 0.5175 | 0.345% |
| 1807 | Chamomile Extract | 1.50 | 1.00% |
| 1805 | Valerian Root Extract | 0.75 | 0.50% |
| 1811 | L-Tryptophan | 1.29 | 0.86% |
| 997 | Sorbitol Solution 70% USP | 60.00 | 28.00% sorbitol, |
| | | | 12% water |
| 995 | Glycerin, USP 99.7% Excipient/ | 33.12 | 22.08% |
| | Food use | | |
| 529 | purified water (Distilled) | 29.9997 | 19.99982% |

79-11 PC78-10 Canine Calming Gel with Pheromone Blend

| RM # | Ingredient | Weight | wt. % |
|---|---|---|---|
| 996 | Yelkin ® Gold - Lecithin | 14.2500 | 9.50% |
| 445 | citation 70 - Mineral oil | 6.0000 | 4.00% |
| 1822 | Pheromone Blend | 0.0009 | 0.0006% |
| 1820 | Beef Ty Fl NAT - Flavoring | 2.2500 | 1.50% |
| 1796 | Melatonin | 0.0645 | 0.043% |
| 1801 | Niacin, USP | 0.2580 | 0.172% |
| 1821 | Thiamine HCl Vitamin B1 | 0.5175 | 0.345% |
| 1807 | Chamomile Extract | 1.5000 | 1.00% |
| 1805 | Valerian Root Extract | 0.7500 | 0.50% |
| 1811 | L-Tryptophan | 1.2900 | 0.86% |
| 997 | Sorbitol Solution 70% USP | 60.0000 | 28.00% sorbitol, |
| | | | 12% water |
| 995 | Glycerin, USP 99.7% Excipient/ | 33.1200 | 22.08% |
| | Food use | | |
| 529 | purified water (Distilled) | 29.9991 | 19.9994% |

80-20 Calming with new rabbit pheromone Tiglinaldehyde

| RM # | Ingredient | Weight | wt. % |
|---|---|---|---|
| 996 | Yelkin ® Gold - Lecithin | 28.5000 | 9.50% |
| 445 | citation 70 - Mineral oil | 12.0000 | 4.00% |
| 1820 | Beef Ty Fl NAT - Flavoring | 4.5000 | 1.50% |
| 1796 | Melatonin | 0.129 | 0.043% |
| 1801 | Niacin, USP | 0.516 | 0.172% |
| 1821 | Thiamine HCl Vitamin B1 | 1.035 | 0.345% |
| 1807 | Chamomile Extract | 3.00 | 1.00% |
| 1805 | Valerian Root Extract | 1.50 | 0.50% |
| 1811 | L-Tryptophan | 2.58 | 0.86% |
| 997 | Sorbitol Solution 70% USP | 120.00 | 28.00% sorbitol, |
| | | | 12% water |
| 995 | Glycerin, USP 99.7% Excipient/ | 66.24 | 22.08% |
| | Food use | | |
| 529 | purified water (Distilled) | 59.99982 | 19.99994% |
| 1877 | Pheromone - tiglinaldehyde | 0.00018 | 0.00006% |

Copy of 76-96 Canine Calming Gel with Quinoline Pheromone

| RM # | Ingredient | Weight | wt. % |
|---|---|---|---|
| 996 | Yelkin ® Gold - Lecithin | 18.80 | 9.40% |
| 445 | citation 70 - Mineral oil | 10.00 | 5.00% |
| 1794 | Hickory Chicken Type FL NAT - | 1.20 | 0.60% |
| | Flavoring | | |
| 1796 | Melatonin | 0.092 | 0.046% |
| 1544 | Quinoline | 0.0002 | 0.0001% |
| 1801 | Niacin, USP | 0.40 | 0.20% |
| 1821 | Thiamine HCl Vitamin B1 | 0.70 | 0.35% |
| 1807 | Chamomile Extract | 9.40 | 4.70% |
| 1805 | Valerian Root Extract | 6.00 | 3.00% |
| 1811 | L-Tryptophan | 1.80 | 0.90% |
| 997 | Sorbitol Solution 70% USP | 80.00 | 28.00% sorbitol, |
| | | | 12% water |
| 995 | Glycerin, USP 99.7% Excipient/ | 24.00 | 12.00% |
| | Food use | | |
| 529 | purified water (Distilled) | 47.6078 | 23.8039% |

### Testing the Samples of Examples 1-11

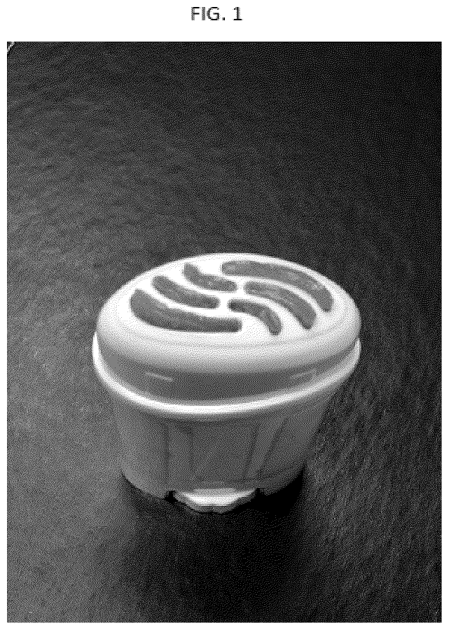

No alcohol, thickeners, fibers or microencapsulated components were present in the gels of Ex-1 through Ex-11. Those components would have made the gel too viscous to administer through the openings of FIG. 1. The calming gel must have low enough viscosity to push through openings that are about ⅛ inch to ¼ inch wide.

In a preferred embodiment, as shown in FIG. 1, the calming gel is placed in a container that has slots or openings at the top. In a preferred embodiment, the openings are about ⅛ to ¼ inch wide. At the bottom of the container is a tightening mechanism that forces the gel up through the openings at the top. The top is smooth and configured to easily fit inside a mouth to apply the gel to the tongue, teeth and gums. In a preferred embodiment, this container is used to apply the gel the mouth, teeth and gums of an animal, such as a dog. As the dog licks the calming gel off the container, the licking motion topically applies the gel to the gums of the dog, where it adheres to the gums and provides a trans-mucosal application of calming ingredients to the gums, and is also ingested as the dog swallows. The calming gel of this invention provides extended release of the calming ingredients. This dual application approach provides superior administration of the calming ingredients to the dog.

The calming gel was eagerly ingested by the test dogs. The smell of quinolone was unpleasant to the human administering the gel, but the dogs were unaffected by it.

The embodiments were chosen and described to best explain the principles of the invention and its practical application to persons who are skilled in the art. As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A method for administering a formulation to an animal, the method comprising:
providing a dispenser containing the formulation, the dispenser comprising:
a top end comprising one or more openings; and
a bottom end comprising a tightening mechanism;
contacting a formulation application area with the top end; and
administering the formulation by adjusting the tightening mechanism to push the formulation through the one or more openings and into contact with the formulation application area.

2. The method of claim 1, wherein the one or more openings include a plurality of openings.

3. The method of claim 1, wherein the formulation application area includes a mouth of the animal.

4. The method of claim 3, wherein the mouth includes an oral cavity, teeth, or gums of the animal.

5. The method of claim 4, wherein the formulation is configured to be ingested by the animal, thereby providing a dual application of the formulation.

6. The method of claim 1, wherein the formulation comprises a gel.

7. The method of claim 6, wherein gel has a viscosity configured to pass through the one or more openings.

8. The method of claim 1, wherein adjusting the tightening mechanism comprises twisting a grip to turn the tightening mechanism.

9. The method of claim 8, wherein the grip has a plurality of rotational and/or clicking positions.

10. The method of claim 9, wherein each rotational position of the plurality of rotational and/or clicking positions corresponds to a dose of the formulation administered through the one or more openings.

11. The method of claim 10, the method further comprising selecting the dose of the formulation based on at least a size of the animal prior to administering the formulation.

12. The method of claim 11, wherein administering the formulation comprises rotating the grip to a rotational and/or clicking position of the plurality of rotational and/or clicking positions, wherein the rotational and/or clicking position corresponds to the dose.

13. A method for administering a formulation to an animal, the method comprising:
providing a dispenser containing the formulation, the dispenser comprising:
a top end comprising one or more openings; and
a bottom end comprising a grip coupled to a tightening mechanism;
selecting a dose of the formulation based on at least a size of the animal;
contacting a formulation application area with the top end; and
turning the grip to a rotational position corresponding to the dose,
wherein turning the grip to the rotational position causes the tightening mechanism to push the dose of the formulation through the one or more openings and into contact with the formulation application area, thereby administering the dose of the formulation.

14. The method of claim 13, wherein the rotational position comprises a plurality of rotational positions.

15. The method of claim 14, wherein each rotational position of the plurality of rotational positions corresponds to a different dose.

16. The method of claim 15, wherein the grip is graduated such that the grip clicks into each rotational position of the plurality of rotational positions.

17. The method of claim 13, wherein the formulation comprises a gel.

18. The method of claim 17, wherein the gel has a viscosity configured to pass through the one or more openings.
